(19) United States
(12) Patent Application Publication
(10) Pub. No.: US 2017/0188892 A1
(43) Pub. Date: Jul. 6, 2017

(54) Method and Apparatus for Using Adaptive Plethysmographic Signal Conditioning to Determine Patient Identity

(71) Applicant: Ivy Biomedical Systems, Inc., Branford, CT (US)

(72) Inventor: Jennifer Laine, Guilford, CT (US)

(73) Assignee: Ivy Biomedical Systems, Inc., Branford, CT (US)

(21) Appl. No.: 15/399,000

(22) Filed: Jan. 5, 2017

Related U.S. Application Data
(60) Provisional application No. 62/274,964, filed on Jan. 5, 2016.

(51) Int. Cl. A61B 5/117; G06F 17/18; G06K 9/00; G01R 13/02; A61B 5/026; A61B 5/1455

(52) U.S. Cl. CPC A61B 5/117; A61B 5/0261; A61B 5/14551; G06K 9/0055; G01R 13/029; G06F 17/18

(57) ABSTRACT

A method for distinguishing between individual human subjects using plethysmographic waveforms. In one embodiment, the method comprises the steps of: receiving a plurality of plethysmographic signals from different individuals; normalizing the signals to the same period length and mean; changing the basis of each of the plurality of signals to obtain its basis vectors and coefficients; reducing the number of coefficients for each signal in the plurality of signals; and calculating the covariance matrix and mean for each individual's set of normalized and dimensionally-reduced plethysmograph waveforms. In another embodiment, the method further comprises the step of taking the Mahalanobis distance between each new plethysmograph waveform that has also been normalized and dimensionally reduced and the stored models of all individuals. In yet another embodiment, the method further comprises the step of finding the index of the stored model with the minimum Mahalanobis distance.

### RELATED APPLICATIONS

This application claims priority to U.S. Provisional Patent Application 62/274,964 filed on Jan. 5, 2016, the contents of which is herein incorporated by reference in its entirety.

### FIELD OF THE INVENTION

The invention relates generally to a method and apparatus to perform pulse plethysmography and more specifically to a method and apparatus for conditioning such plethysmographic signals to obtain other patient information, such as patient identity.

### BACKGROUND OF THE INVENTION

Plethysmography is the measurement of change in the volume of an organ or a body. Pulse plethysmography is the measurement of change in a volume of a blood vessel as a bolus of blood moves through the vessel with each heartbeat. The measurement of vessel volume fluctuation with each pulse is useful in non-invasively determining various other physiologic parameters, such as blood pressure and cardiac ejection volume.

Pulse oximetry is utilized to determine the oxygen levels in the blood, typically by measuring the absorption of various wavelengths of light by blood passing through the vessels of the skin. Pulse oximetry information is very useful because it is a non-invasive and potentially omnipresent way of obtaining blood oxygen levels and other circulatory information. Pulse oximetry measurements are also affected by blood vessel volume changes and therefore can be used to obtain plethysmographic information.

Each person has a unique vascular structure and cardiac cycle that can be characterized by the structural cues of the plethysmograph signal, and more information is embedded in this signal than merely the blood oxygen saturation value. No two people have exactly the same plethysmograph waveform when measured with sufficient detector sensitivity.

A system that adaptively learns the intrinsic geometry of each subject's waveforms from labeled training examples could be used as an identification tool and would add further value to patient monitoring and be a foundation for further applications.

What is needed is a method and apparatus to provide such discrimination. The present invention addresses this need.

### SUMMARY OF THE INVENTION

In one aspect, the invention characterizes the subtle structural differences in the plethysmograph wave between individuals. In one embodiment, the method comprises the steps of: receiving a plurality of plethysmographic signals; standardizing the signals to the same period length and mean; numerically normalizing the signals to values between 0 and 1; changing the basis of each of the plurality of signals to optimize a metric that minimizes the distance between data of the same person while maximizing the distance between data of different people; reducing the number of coefficients in the projection of each signal along the new basis. In another embodiment, the method further uses the aforementioned information for creating a model for each individual for comparison to future plethysmograph waveforms.

In another aspect, the invention relates to an apparatus for distinguishing individuals based purely on their plethysmographic waveforms. In one embodiment, the apparatus comprises a source of plethysmographic signals; a processor in communication with the source of plethysmographic signals; and a database in communication with the processor. In one embodiment, the processor receives a plurality of plethysmographic signals from the source of plethysmographic signals. In another embodiment, the processor: standardizes the plethysmographic signals to the same period length and mean; numerically normalizes the signals to values between 0 and 1; changes the basis of each of the plurality of plethysmographic signals to obtain its data-driven basis vectors and coefficients; reduces the number of coefficients for each plethysmographic signal in the plurality of signals; and creates a sparse model of each individual based on the Mahalanobis distance metric. In still another embodiment, the source of the signal is selected from the group including pressure transducers, optical transducers, impedance electrodes, acoustic transducers, or other physiologic transducers.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The structure and function of the invention can be best understood from the description herein in conjunction with the accompanying figures. The figures are not necessarily to scale, emphasis instead generally being placed upon illustrative principles. The figures are to be considered illustrative in all aspects and are not intended to limit the invention, the scope of which is defined only by the claims.

FIG. 1 is a block diagram of an embodiment of a system for practicing the invention.

FIG. 2 is a flow diagram and visualization aid of an embodiment of a method to identify a specific individual from a group of individuals using a plethysmographic period.

FIGS. 3A and 3B is a flow chart of an embodiment of the training of the classifier with randomly selected training periods from subjects, and consequent validation of the basis selection and metric with separate validation data.

FIG. 4A is a plurality of graphs of the raw plethysmographic periods (IR) from a number of individuals superimposed upon one another.

FIG. 4B is a plurality of graphs of the plethysmographic periods of FIG. 4A superimposed after pre-processing.

FIG. 5 is a 2D scatter plot visualization of the pre-processed plethysmograph periods along the first two basis vectors distinguished by color associated by subject. In the embodiment shown, there were 59 subjects in total.

### DESCRIPTION OF A PREFERRED EMBODIMENT

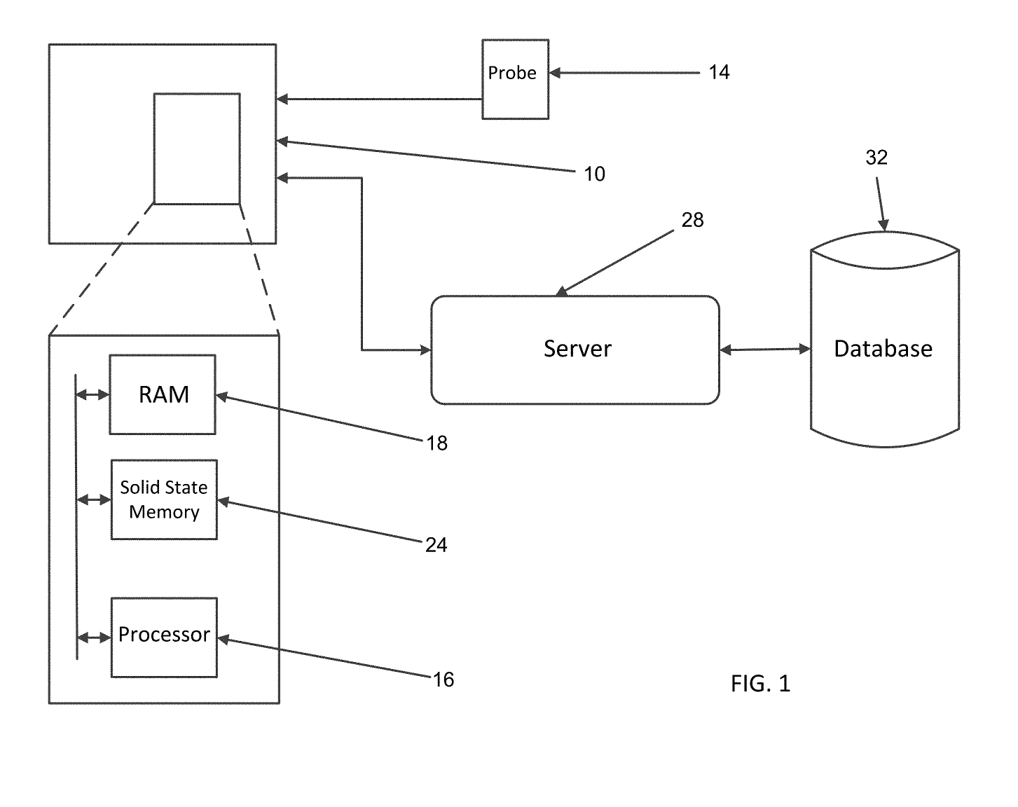

Referring to FIG. 1, in one embodiment, a system 10 used to generate data in accordance with the invention includes an optical finger probe 14. The finger probe 14 includes a light source and a detector. Reflectance measurements by the detector are used by a processor 16 to measure the variations in the blood flow through the finger. In one embodiment, data input from the finger probe 14 and the results of the reflectance calculations performed by the processor 16 are stored first in RAM memory 18 and eventually stored in disk or solid state memory 24 within the device housing. Alternatively, the raw data and results of the calculations are transferred directly to a server 28 for storage in mass storage 32. The server 28 may then make various statistical calculations on the data. Other methods of plethysmographic signal generation such as measurements using a continuous digital pressure cuff may be used to provide data to processor 16.

In one embodiment, the device used to acquire data is an oximeter. Normally, an oximeter includes a finger probe 14 that includes two light sources of different wavelengths and two detectors. Reflectance measurements by the detectors of the oximeter are used by the processor 16 to calculate the amount of oxyhemoglobin in the blood using the methods known to the art. Generally, as the blood pulses through the arteries, the pulsations will register as a change in reflectance as a result in the change in the light absorption during the pulse. Ordinarily, this pulsation is similar to a shift in baseline and must be compensated for such that the absorption by the blood can be measured as a stable value indicative of the amount of oxyhemoglobin in the patient's blood. In the present invention, it is the measurement of these pulsations that is important. Other devices to measure pulsations such as pressure transducers, optical transducers, impedance electrodes, acoustic transducers, or other physiologic transducers may be used instead of the oximeter.

In the present embodiment, the data from only one light source of the two light sources and one detector of the two detectors in the finger probe of the oximeter are used. No actual measure of oxyhemoglobin is made, and the data collected are the fluctuations in absorbance or reflectance caused by the pulsations of the blood moving through the arteries. These fluctuations are then the plethysmographic waveforms input into the processor of the invention for processing.

Briefly, the processing of the signal takes place in two phases. The first phase includes the training of the system to transform the plethysmograph space into a feature space that maximizes differences between periods of different people using a data-driven metric in which the metric is determined by the data being measured. The second phase is the validation of the space transformation by measuring the distance between test data from the same individuals and their trained sparse representations. Subsequently, a future waveform from a catalogued individual may be similarly transformed and compared against the validated representations to identify the future waveform as belonging to the respective individual.

In practice, reflectance periods, or arterial blood oxygen saturation (SpO2) periods, in the case of an oximeter, have variations in the number of data points in the measurement and the mean of their values. These two parameters must be standardized first before subsequent processing so that the analysis focuses on the slight deviations in the structure of the waveform instead of the offset and period length differences between various samples.

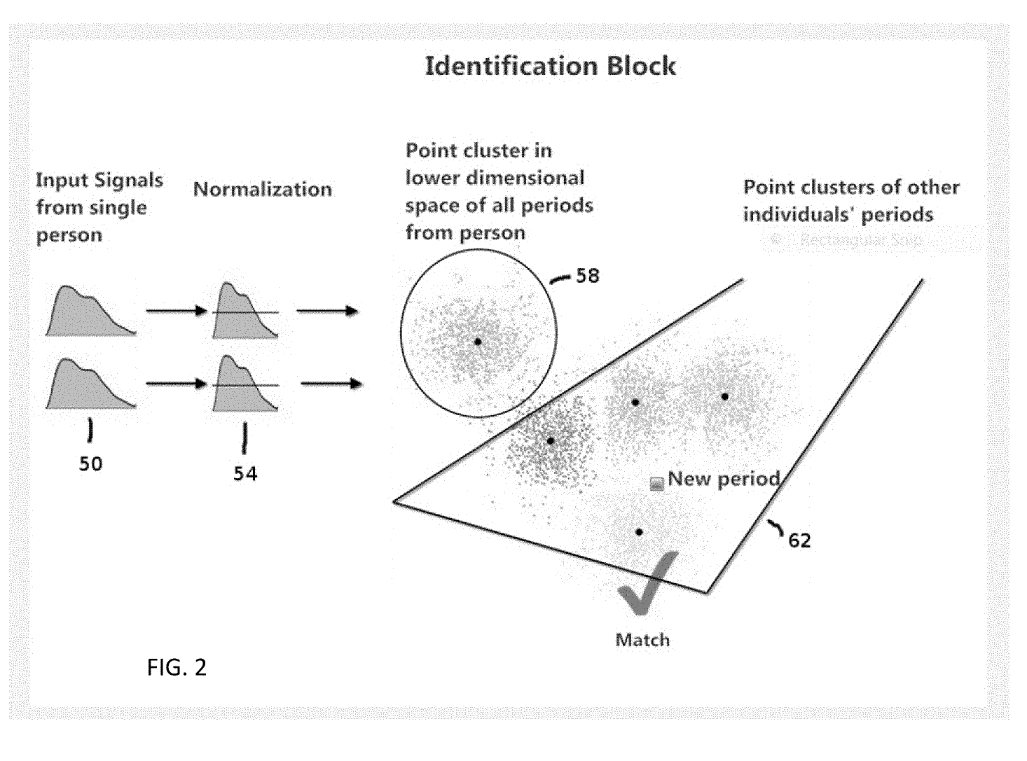

Referring to FIG. 2, visualization of the method of the invention is shown in more detail. The system is first trained to identify a representative plethysmographic waveform period from one individual and distinguish that from plethysmographic waveform periods from other individuals. The training begins by recording the input signal (Step 50), and standardizing the signal to a uniform period length and mean, while spanning the values [0,1] (Step 54). The method then changes the basis of the space while removing the higher dimensional coefficients to reduce dimensionality and optimize the efficacy of the data-driven metric (Step 58). The number of coefficients retained is determined empirically by examining how many matches are obtained for a given number of coefficients remaining. The method also incorporates the shape of the distribution of the training observations of each individual in the new space into the distance metric calculation. The learned statistical models of various individuals' coefficients in the new space are stored in a database 62. Subsequent data taken from the individual to be identified are similarly pre-processed and transformed to the new space, compared to the models of all individuals in the database 62 using the metric trained for each individual. The model in the database having the minimum distance metric of the group of models is considered the best match. If each individual's model in the database is represented by an index, then a plurality of periods from an individual is matched to a model by taking the index of maximum cardinality or mode of the set of best match indices. The ability to distinguish one individual's plethysmographic periods from another individual's plethysmographic periods has many applications, including the trending of a human subject's medical state and biometrics.

Considering the individual steps in the process in more detail, the method relies on the ability of the system to effectively cluster plethysmograph wave data belonging to the same individual so that future additional waveforms from that individual may be classified correctly. To reduce data analysis and memory constraints during both training and testing, a sparse representation of the primary characteristics of a plethysmographic signal period may be desirable.

The present embodiment uses an orthogonal basis that spans the entire data point space and also pinpoints effectively the differences between the plethysmographic features of different individuals. Randomly chosen bases that were empirically optimized worked best for the chosen supervised learning method.

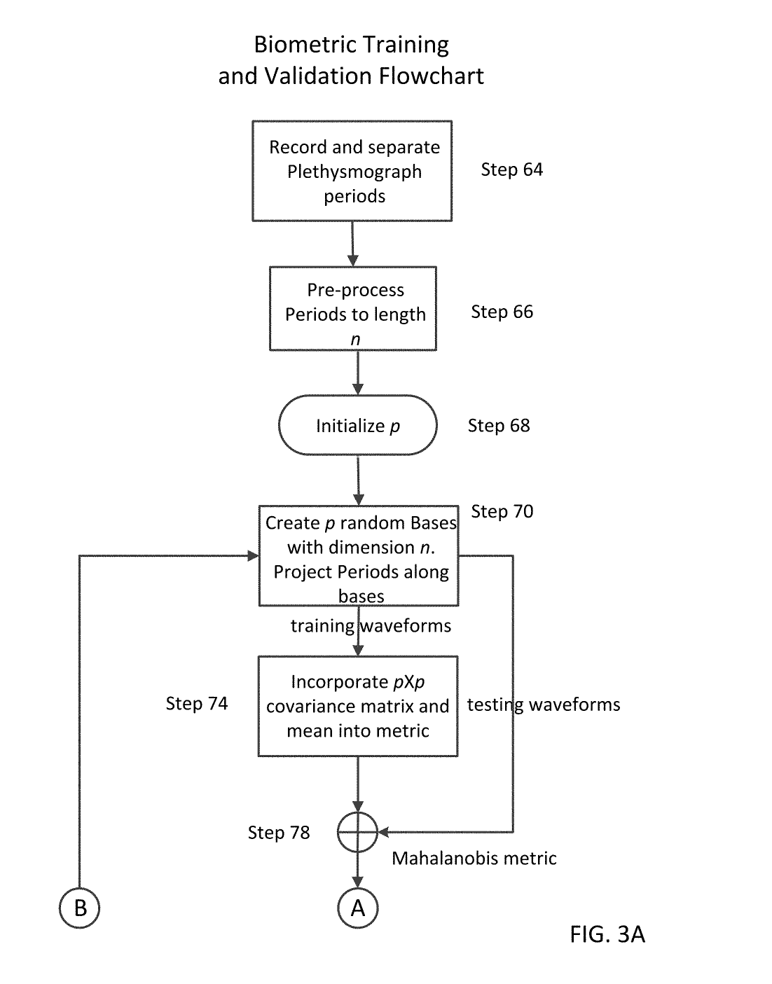

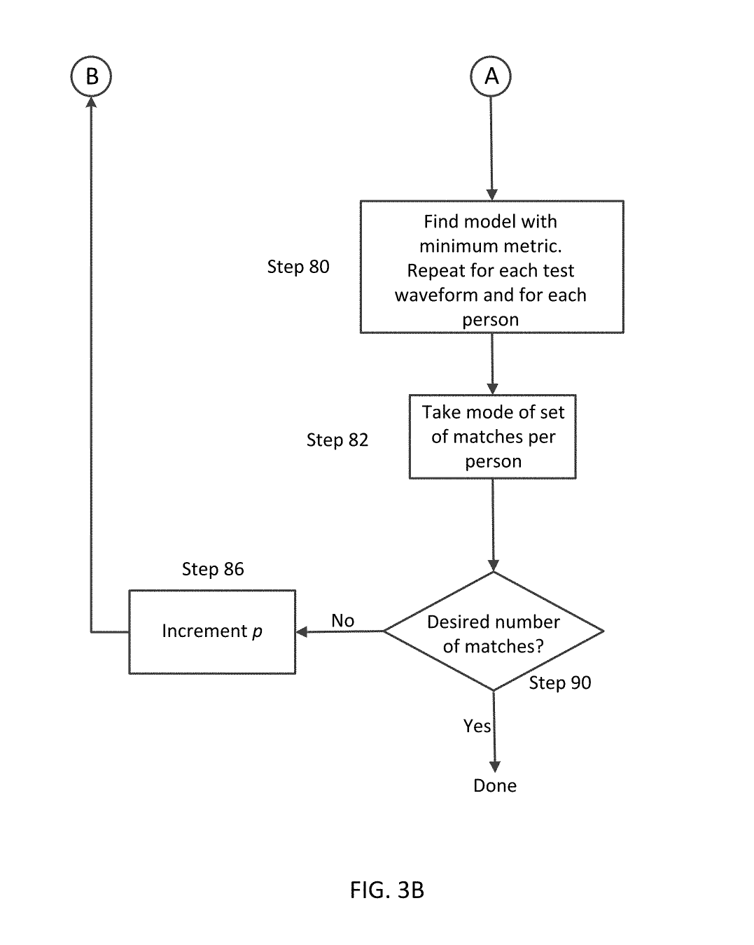

As part of the training section of FIGS. 3A and 3B, in one embodiment, nearly 22,000 plethysmographic periods using an infrared (IR) wavelength for measurement were captured (Step 64) from 59 human subjects. Other numbers of periods and human subjects could be used. Each observation or period was pre-processed in the following way. First, the waveform was standardized by taking the mean to zero, contracted or dilated to the average period length, and numerically normalized to take on values between 0 and 1 (Step 66). An original observation, X,

X={xᵢ}ᵢ₌₁ⁿ

which is a collection of data points, where n is the number of samples per pre-processed period, was transformed down to a smaller dimension p by first initializing index p to zero (Step 68) and then at each iteration, projecting each period along p specifically chosen bases (Step 70).

{φᵢ}ᵢ₌₁^{p}

where φᵢ is the basis.
The resultant waveform in the new feature space is X*.

X*=Σᵢ₌₁^{p}(X·φᵢ)φᵢ (1)

The number of samples in the waveform in the new feature space is decreased from n (in the prior feature space X) to p. These samples or coefficients of the observation are the dot products (X·φᵢ) for all i from 1 to p. The number of bases is chosen empirically until a user-defined, pre-determined threshold is met after the application of an iterative Monte Carlo approach of randomly varying p. The threshold is selected in part by the amount of time allocated by the user for making the calculations, the number of subjects in the study, and the number of measured periods per subject available for training. In one embodiment, the value of p=6. The value of n was the average length of all periods examined and was equal to n=764.

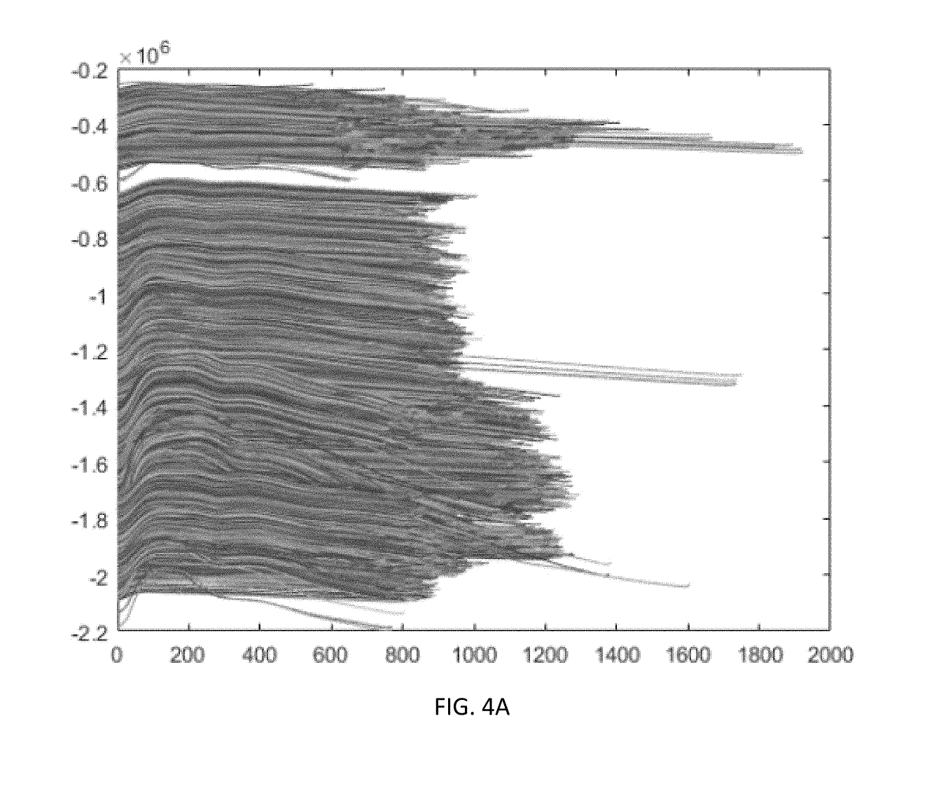

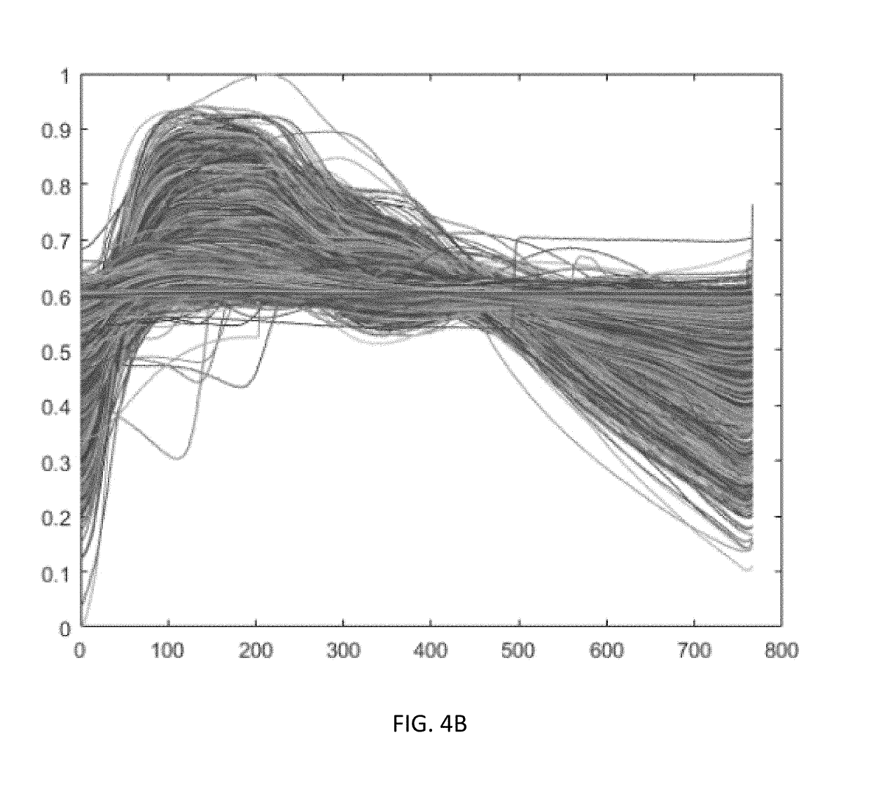

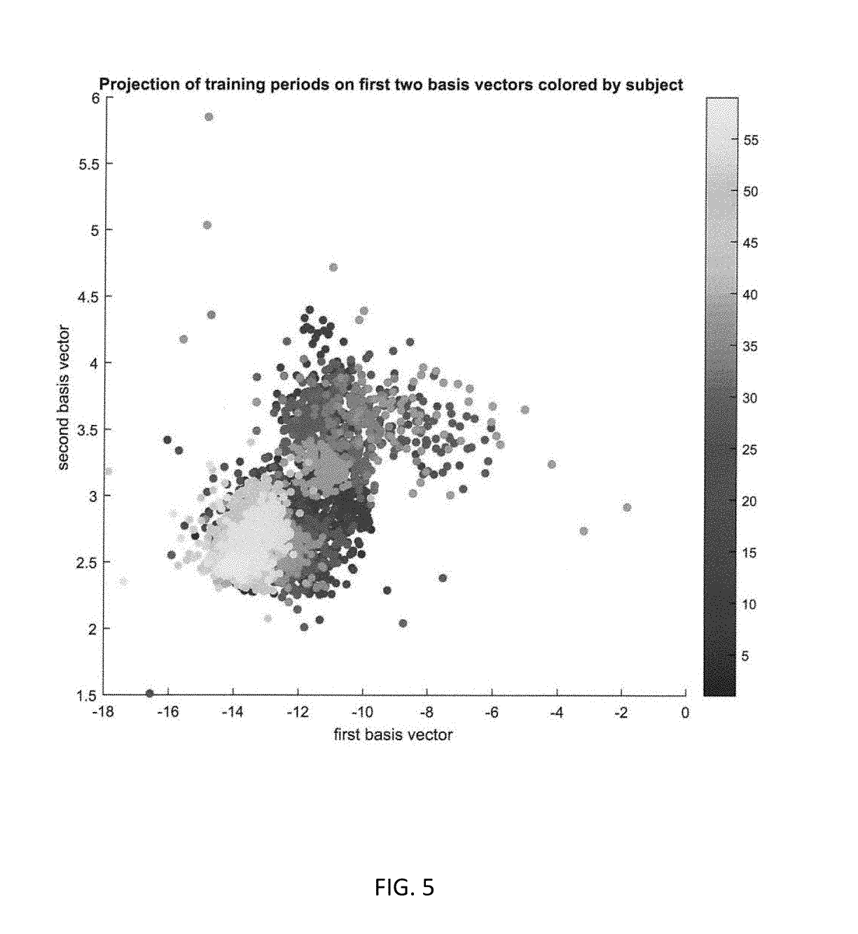

In one embodiment, half of the pre-processed periods were used to train the system, and half were used as test waveforms. FIGS. 4A and 4B show both testing and training observations (periods) superimposed upon one another, both before pre-processing (FIG. 4A) and after pre-processing (FIG. 4B). In FIG. 5, the first two coefficients of all transformed training periods exemplified by the first two dot products of Equation (1) are plotted against each other, and each resulting data point is assigned a color by subject.

Referring again to FIGS. 3A and 3B, the covariance matrix for this method is a square matrix whose size is #(data points per period)×#(data points per period) or in this case p×p. It represents the general shape of the distribution of the training data from a single individual in the new feature space X* having p dimensions. A representative model of each individual's waveforms was constructed using the covariance matrix and mean waveform of all the training data from that individual (Step 74). The dimension of the covariance matrix and mean were varied through the iterative approach of varying p to find the representation that gave the best results for the data. The distance between a new waveform observation from the testing waveform suite and the data distribution of each stored individual was calculated using the Mahalanobis distance (Step 78). The metric provides the notion of nearness between an unclassified plethysmograph period and the distribution of representative observations from the same person and different people (Step 80).

Defining Jᵢ as the collection of all pre-processed and dimensionally transformed periods in the testing data for person I, then,

Jᵢ={X*ₖ}ₖ₌₁^{N}^{ᵢ}

where Nᵢ is the number of transformed test periods from person i that are measured against the trained models. All Nᵢ periods from person i's test data are compared to the distributions of all modeled individuals. A match is made when the Mahalanobis distance between test period X*ₖ for person i and a target individual j is a minimum. If each stored model has an index j associated to it, then the matching index mₖⁱ (the index having the minimum distance to the new period being examined) is determined thus:

$${m}_{k}^{i} = arg {min}_{j} ( \sqrt{( {X}_{k}^{*} - {μ}_{j} ) {{C}_{j}^{- 1} ( {X}_{k}^{*} - {μ}_{j} )}^{T}} )$$

where μⱼ is the mean of person j's training periods, and Cⱼ⁻¹ is the inverse of the covariance matrix of person j's training data. The result is a set Mᵢ of Nᵢ matching indices for all test periods of person i. The resulting classification label lᵢ for the testing waveforms of person i is determined by taking the index with the maximum number of matches in Mᵢ (Step 82).

lᵢ=mode(Mᵢ)

As referred to in FIGS. 3A and 3B (Step 86), the number of coefficients accessed in the training and testing waveforms was adjusted and the process repeated until a desired number of correct matches occurred (Step 90).

In one test, 59 subjects were modeled individually by their plethysmograph pulses in the training suite. The time-series recording of each individual spanned between 0.5 minutes and 27 minutes. Randomly selected periods of each person's data amounting to half of the recording were used to train each person's respective model, and the other half were used as test data. As few as 15 periods and as many as 800 periods per individual were used to both train the model and also test the algorithm. In one embodiment of the 59 individuals, 58 of them (98.3%) were correctly identified. Of the group of 59 subjects, one individual was incorrectly matched 11 times, and correctly matched 4 times. Therefore, the model based on a small number of periods, in this case 15, was probably insufficient. In this example, 15 periods was the minimum used in the whole ensemble for training.

Unless otherwise indicated, all numbers expressing lengths, widths, depths, or other dimensions and so forth used in the specification and claims are to be understood in all instances as indicating both the exact values as shown and as being modified by the term “about.” Accordingly, unless indicated to the contrary, the numerical parameters set forth in the specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques. Any specific value may vary by 20%.

The terms “a,” “an,” “the,” and similar referents used in the context of describing the invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of any claim. No language in the specification should be construed as indicating any non-claimed element essential to the practice of the invention.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified, thus fulfilling the written description of all Markush groups used in the appended claims.

Certain embodiments are described herein, including the best mode known to the inventor for carrying out the spirit of the present disclosure. Of course, variations on these described embodiments will become apparent to those of ordinary skill in the art upon reading the foregoing description. The inventor expects skilled artisans to employ such variations as appropriate, and the inventor intends for the invention to be practiced otherwise than specifically described herein. Accordingly, the claims include all modifications and equivalents of the subject matter recited in the claims as permitted by applicable law. Moreover, any combination of the above-described elements in all possible variations thereof is contemplated unless otherwise indicated herein or otherwise clearly contradicted by context.

In closing, it is to be understood that the embodiments disclosed herein are illustrative of the principles of the claims. Other modifications that may be employed are within the scope of the claims. Thus, by way of example, but not of limitation, alternative embodiments may be utilized in accordance with the teachings herein. Accordingly, the claims are not limited to embodiments precisely as shown and described.

What is claimed is:

1. A method for distinguishing individuals based purely on their plethysmographic waveforms comprising the steps of:
receiving a plurality of plethysmographic signals;
standardizing the plethysmographic signals to the same period length and mean;
numerically normalizing the signals to values between 0 and 1;
changing the basis of each of the plurality of plethysmographic signals to obtain its data-driven basis vectors and coefficients;
reducing the number of coefficients for each plethysmographic signal in the plurality of plethysmographic signals; and
creating a sparse model of each individual based on a Mahalanobis distance metric.

2. The method of claim 1 further comprising the step of comparing new plethysmograph waveforms from an individual to the database of stored models.

3. The method of claim 2 further comprising the step of calculating the mode of an individual's collection of matched indices having the minimum Mahalanobis distance between its stored individual's model and the new plethysmograph periods.

4. The method of claim 3 further comprising the step of adjusting or incrementing the number of basis vectors until a desired number of correct matches is achieved.

5. The method of claim 1 wherein the source of the signal is selected from the group including pressure transducers, optical transducers, impedance electrodes, acoustic transducers, or other physiologic transducers.

6. The method of claim 5 wherein the optical transducer is infrared.

7. An apparatus for distinguishing individuals based purely on their plethysmographic waveforms comprising:
a source of plethysmographic signals;
a processor in communication with the source of plethysmographic signals; and
a database in communication with the processor,
wherein the processor receives a plurality of plethysmographic signals from the source of plethysmographic signals;
wherein the processor:
standardizes the plethysmographic signals to the same period length and mean;
numerically normalizes the signals to values between 0 and 1;
changes the basis of each of the plurality of plethysmographic signals to obtain its data-driven basis vectors and coefficients;
reduces the number of coefficients for each plethysmographic signal in the plurality of signals; and
creates a sparse model of each individual based on the Mahalanobis distance metric.

8. The apparatus of claim 7 wherein the source of the signal is selected from the group including pressure transducers, optical transducers, impedance electrodes, acoustic transducers, or other physiologic transducers.
